(19) United States
(12) Patent Application Publication
(10) Pub. No.: US 2004/0248858 A1
(43) Pub. Date: Dec. 9, 2004

(54) Cure for cancer

(76) Inventor: Kaumil H. Parghi, Tempe, AZ (US)

Correspondence Address: PARGHI, KAUMIL H., 1019 E. LEMON ST., # 105, TEMPE, AZ, 85281, US

(21) Appl. No.: 10/457,714

(22) Filed: Jun. 9, 2003

(51) Int. Cl. A61K 31/60

(52) U.S. Cl. 514/159

(57) ABSTRACT

The invention provides for application of Salicylic Acid, in proportion and concentration depending upon the size of cancer, onto or into a cancerous area in the body, in order to reduce and remove the cancer. Upon application, Salicylic Acid will destroy all the extra cells in the body that form cancer and rid the body of cancer. Salicylic Acid is the cure for cancer.

### CROSS-REFERENCE TO RELATED APPLICATIONS

Not Applicable

### STATEMENT RE: FEDERALLY SPONSORED RESEARCH OR DEVELOPMENT

Not Applicable

### REFERENCE TO A MIRCROFICHE APPENDIX

Not Applicable

### BACKGROUND OF THE INVENTION

Intake of proper food has prevented humans from developing cancer. However, since humans do not know what chemical it is that prevents cancer, they have not been careful in eating foods that have this chemical. Discovering this chemical and eating foods that have this chemical will prevent cancer. Application of this chemical in proper proportion and concentration, to a cancerous area will result in reduction and removal of cancer.

### SUMMARY OF THE INVENTION

The chemical that prevents and cures cancer is Salicylic Acid. Salicylic Acid, if applied directly onto, or injected into the affected area, in proportion and concentration depending upon the size of cancer, will reduce and remove cancer of all types, including leukemia, or blood cancer. A 40% solution of Salicylic Acid is effective in reducing and removing small pimple sized cancerous cells. Higher concentrations and doses would be needed for application on cancer areas depending on their size and density, for their removal.

### DESCRIPTION OF THE INVENTION

Eating foods that have proper proportions and concentration of Salicylic Acid in them will prevent cancer formation. Fruits and vegetables have Salicylic Acid in them in various proportions. Therefore, eating a diet with fruits and vegetables in them prevents hemorrhoids and colon cancer.

Salicylic Acid, if applied directly onto, or injected into the affected area, in proportion and concentration depending upon the size of cancer, will reduce and remove cancer of all types, including leukemia, or blood cancer. A 40% solution of Salicylic Acid is effective in reducing and removing small pimple sized cancerous cells. Higher concentrations and doses would be needed for application on cancer areas depending on their size and density, for their removal. The application of Salicylic Acid can be in form of cream that can be rubbed onto the affected area if the area is on the surface of the body. Or, the application of Salicylic Acid can be in form of liquid solution that can be injected into a tumor inside the body, like a brain or a breast tumor. Similarly, Salicylic Acid can be injected in form of a liquid into the blood stream of patients that have blood cancer or leukemia. Upon application, Salicylic Acid will destroy all the extra cells in the body that form cancer and rid the body of cancer.

1. I claim that Salicylic Acid, if applied directly onto, or injected into the affected area, in proportion and concentration depending upon the size of cancer, will reduce and remove cancer of all types, including leukemia, or blood cancer.
